(19) United States
(12) Patent Application Publication
(10) Pub. No.: US 2018/0116611 A1
(43) Pub. Date: May 3, 2018

(54) INSTRUMENT FOR MEASURING STRUCTURAL CHANGE AND DAMAGE IN LUPUS

(71) Applicant: Ketan Desai, Easton, PA (US)

(72) Inventor: Ketan Desai, Easton, PA (US)

(21) Appl. No.: 15/342,465

(22) Filed: Nov. 3, 2016

(51) Int. Cl. A61B 6/03; A61B 8/08

(52) U.S. Cl. CPC A61B 6/032; A61B 8/08; A61B 6/037

(57) ABSTRACT

A method of quantifying structural damage in Lupus is described. The method can be used to assess progress in clinical trials or in clinical practice.

This application claims priority to U.S. Provisional Application No. 62/262,478, the contents of which are hereby incorporated by reference in its entirety for all purposes.

### BACKGROUND OF THE INVENTION

Systemic Lupus Erythematosus (SLE) is an auto-immune disease characterized by multi-system organ involvement. The pathogenesis is not known and treatment is with immunomodulators. Despite best efforts, most patients have organ damage which in the end proves fatal. No radiological instrument exists to quantify that damage or to show a change in organ damage over time. This patent describes SMILE—Structural Modification in Lupus Erythematosus, an instrument that quantifies organ damage.

### SUMMARY OF THE INVENTION

SMILE is a quantitative instrument that gives a numerical value to a Lupus patient's organ damage at a time point and compares it to previous time points. It can be used to demonstrate improvement or worsening of the damage over a period of time. For example, a patient with organ damage will receive a score at an imaging session. A repeat exam using the same imaging instrument (X ray, PET scan, CT scan, MRI, or ultrasound) will similarly give a score at a later time point. Comparison of the scores will give evidence of worsening or improvement of organ damage. Thus, it provides an objective assessment of a patient's status that can guide therapy. When used in clinical trials, it can provide objective evidence of drug effect on structure as opposed to only subjective improvement. The imaging can be done in intervals of months or years.

### DETAILED DESCRIPTION

Lupus is one of many disorders of the immune system known as autoimmune diseases. In autoimmune diseases, the immune system turns against parts of the body it is designed to protect. This leads to inflammation and damage to various body tissues. Lupus can affect many parts of the body, including the joints, skin, kidneys, heart, lungs, blood vessels, and brain. Although people with the disease may have many different symptoms, some of the most common ones include extreme fatigue, painful or swollen joints (arthritis), unexplained fever, skin rashes, and kidney problems.

Diagnosis of lupus is often difficult and the warning signs of lupus can mimic the warning signs of other diseases. Common symptoms of lupus include persistent low-grade fever, extreme fatigue, and painful or swollen joints. The so-called lupus rash, which often manifests as a butterfly-shaped reddish or purplish rash across the bridge of the nose and cheeks, is another common medical sign. However, no single test can be used to diagnose lupus, and it may take several months or years after symptoms first appear for doctors to make a definitive diagnosis. There are blood tests that a doctor can use to help diagnose lupus, but none of these tests are definitive.

More commonly, SLE is a chronic, life-long disease, alternating between periods of symptom relapse, (called flares), and remission. The disease may begin in any of the various systems of the body and progress unpredictably to others. The following are typical patterns:
Symptom relapses, or flares, occur on the average of two or three times a year.
Between flares, most patients with SLE function at about 90% of normal capacity.

The degree of severity depends on different factors:
Severity of the inflammatory response
Frequency of episodes
The degree of organ or system involvement

Vital organs or systems, such as lungs, kidneys, nervous system, joints skin, and others are affected in over a half of patients with SLE. Infections followed by kidney failure are the chief causes of death in patients with SLE.

Because of more effective and aggressive treatment, the prognosis for SLE has improved markedly over the past two decades. Treatment early in the course of the illness improves long-term progress. About 85-95% of people with lupus survive 10 years, and many people have a normal life span. SLE that develops later in life is generally less serious than SLE that strikes in childhood or young adulthood.

As mentioned, there is organ damage in patients with Lupus. Quantifying this organ damage could be very useful in guiding therapy as there could be organ damage in the absence of ongoing symptoms. Therapy could be adjusted to prevent organ damage. Quantifying organ damage could also be useful in clinical trials to provide objective evidence of response, as is done Western Ontario and McMaster Universities Arthritis Index (WOMAC), a trademarked and proprietary index used in Osteoarthritis.

This invention describes such a quantitative tool, Structural Modification in Lupus Erythematosus, or SMILE. By the use of imaging techniques, which could be total body conventional X ray, PET scan, MRI, CT scan or ultrasound, the whole body can be imaged. The imaging is done as is done normally for that modality. Organ damage is identified by reviewing the images and then identified on the table below. A numerical value given for each organ damage. The items on the left side of the table identify the organ and feature involved. The columns on the right demonstrate its relative importance by assigning a numerical value. The numerical value is ascribed as 0 (damage absent), 1 (present), 2 (present, multiple lesions as in hemorrhage, cardiovascular infarction, GI infarction below duodenum, resection of bowel below duodenum, avascular necrosis). For a lesion, the value can be 1 or 2, but not both. For example, for avascular necrosis, either there is one lesion only (thus a score of 1) or more than one lesion (score of 2). Presence of any renal occlusive disease gets a score of 2. The minimum total score is 0, the maximum is 41. The total value is summed up and the value stored in patient's chart along with the images.

The patient has a repeat visit in weeks or months, or even years. The same evaluation is done using the same imaging technique to provide a fair comparison. Each value (line item) can increase or decrease and the total score be appropriately affected. The total value is summed up again and compared to the previous one. This will provide evidence of improvement or deterioration in the organs imaged.

TABLE 1

| Malignancy | |
|---|---|
| Hepatobiliary | 1 |
| Lymphoma | 1 |
| Lung | 1 |
| Central Nervous System | |
| Atrophy | 1 |
| Ischemia | 1 |
| Hemorrhage (single) | 1 |
| Hemorrhage (multiple) | 2 |
| Renal | |
| Shrinkage of bladder | 1 |
| Shrinkage of kidneys | 1 |
| Enlarged kidneys | 1 |
| Vasculitis | 1 |
| Occlusive disease | 2 |
| Pulmonary | |
| Fibrosis | 1 |
| Shrinking lung | 1 |
| Embolism | 1 |
| Interstitial Lung Disease | 1 |
| Infarct | 1 |
| Cardiovascular | |
| Myocarditis | 1 |
| Pericarditis | 1 |
| Infarction (single) | 1 |
| Infarction (multiple) | 2 |
| Coronary artery disease | 1 |
| Valvular disease | 1 |
| Endocarditis | 1 |
| Peripheral Vascular | |
| Minor pulp space loss | 1 |
| Vasculitis | 1 |
| Venous thrombosis | 1 |
| Gastrointestinal | |
| Peritonitis | 1 |
| Vasculitis | 1 |
| Mesentric Insufficiency | 1 |
| Infarction of bowel below duodenum (single) | 1 |
| Infarction of bowel below duodenum (multiple) | 2 |
| Resection of bowel below duodenum (single) | 1 |
| Resection of bowel below duodenum (multiple) | 2 |
| Musculoskeletal | |
| Tenosynovitis | 1 |
| Osteomyelitis | 1 |
| Avascular Necrosis (single) | 1 |
| Avascular Necrosis (multiple) | 2 |
| Myositis | 1 |
| Non erosive arthritis | 1 |

Total:
To give an example of how this works, a patient could have asymptomatic shrinkage of bladder (value of 1), shrinkage of kidneys (value of 1), pericarditis (value of 1) and myositis (value of 1) detected by total body MRI to give a total value of 4. In six months, a repeat total body MRI scan shows no myositis or pericarditis or any other lesion. Thus the score is 0 and the patient has improved.
To give another example, a patient could have osteonecrosis of a single joint for a total value of 1 by X rays. In a year, X rays shows avascular necrosis in an additional joint to give a total value of 2. The score has worsened and aggressive therapy is warranted.

Treatment of Lupus

SMILE can be used to provide objective evidence of improvement or deterioration in the patient's condition and therapy altered accordingly. The patient's baseline value is compared to the value at a follow up visit, which could be in weeks, months or years.

Clinical Trials in Lupus.

SMILE can be used to provide objective evidence of improvement or deterioration in the clinical trial participant who could be on active drug or relevant comparator. The participant's baseline value is compared to the value at a follow up visit, which could be in weeks, months or years. Cohort analysis of participants would give an objective evidence improvement or deterioration of participants in that cohort and influence further development or approval of the drug.

What is claimed:

1. Method of use of tabulating and scoring structural damage in Lupus using radiology.

2. Method of use described in claim 1 where radiology technique is PET scan, MRI, ultrasound, CT scan, or X ray.

3. Method of use described in claim 1 where SMILE is used to quantify structural damage.

4. Method of use described in claim 1 where SMILE is used to show improvement or worsening of organ damage over time

5. Method of use described in claim 1 where SMILE is used in clinical practice to quantify organ damage and influence therapy

6. Method of use described in claim 1 where SMILE is used in clinical research to quantify organ damage
